(19) United States
(12) Patent Application Publication
(10) Pub. No.: US 2005/0070780 A1
(43) Pub. Date: Mar. 31, 2005

(54) Field alterable medical imaging technology

(75) Inventors: Frank D'Amelio, Los Olivos, CA (US); Erhan Gunday, Memphis, TN (US)

Correspondence Address: GANZ LAW, P.C., P O BOX 2200, HILLSBORO, OR, 97123, US

(73) Assignee: Circon Corporation

(21) Appl. No.: 10/675,905

(22) Filed: Sep. 29, 2003

(51) Int. Cl. A61B 5/05; A61B 6/00

(52) U.S. Cl. 600/407; 600/476

(57) ABSTRACT

A medical imaging system including one or more medical devices, operational electronics associated at least one medical device, embedded software associated with operational electronics, and an alteration mechanism providing alteration of at least one of operational electronics and embedded software. In one aspect, a medical device has a configurable component and/or configuring software and has an alteration mechanism that provides alteration of the configurable component and/or the configuring software. In another aspect, a medical device has a programmable component and/or programming software, and has an alteration mechanism that provides alteration of the programmable component and/or the programming software.

### BACKGROUND

The present invention relates generally to medical imaging technology and, more particularly, to medical imaging technology providing systems, devices and methods having field alterable electronics.

Minimally invasive surgery generally reduces patients' surgery-induced trauma and pain, promotes and accelerates recovery and, by limiting hospitalization, helps control health care costs. Accordingly, minimally invasive surgery is of ever-growing importance, as are the systems, devices and methods associated therewith.

Minimally invasive surgery typically contemplates the employ of medical imaging systems in combination with surgical instruments, such as graspers, dissectors, scissors, biopsy instruments, and needle holders. Medical imaging systems generally comprise an optical instrument (e.g., an endoscope) providing an image to a video camera. A typical video camera comprises operational electronics, including a video sensor (such as a CCD or conventional video camera) by which an image is acquired as an electronic signal. A typical video camera's operational electronics also tends to include control interfaces (e.g., buttons, LEDs, etc.), which may be variously disposed (e.g., on a controller and/or on a camera head), and which are applicable in receiving and handling—and, generally, processing—one or more acquired signals, as well as to provide one or more output or feedback signals. As well, a typical video camera's operational electronics also tends to include one or more programmable components (e.g., a microprocessor) and/or one or more configurable components (e.g., Field Programmable Gate Array (“FPGA”) and Programmable Logic Device (“PLD”)), as well as other components (e.g., a memory system).

Medical imaging systems generally comprise other devices having operational electronics. One such device may be, for example, an insufflator, used for distention of the abdominal cavity for laparoscopic procedures, through introduction of distention fluids.

Video cameras and other devices in medical imaging systems generally have embedded software that, in combination with the operational electronics, determines the device's functions. This software generally may include, without limitation, programs, directives, commands, processes, routines, information or other programming code and/or data to be executed or otherwise similarly used by a microprocessor or other programmable components. Such software generally may also include, without limitation, constants, look-up table data, information, directives, commands, routines or other configuring code and/or data to be loaded into or otherwise used to configure a FPGA or other configurable components. Such software can be variously embedded, including by storing it in a memory system, implementing it as firmware and/or loading it in to configure one or more configurable components.

A video camera's embedded software and operational electronics conventionally are implemented at the time of manufacture. As such, embedded software and operational electronics generally cannot be readily altered in the field, particularly by a surgeon or other similar end user. Alteration of the embedded software can include one or more of replacement, supplement, update, upgrade, debugging, compaction, or any other change to any code and/or data, with or without improvement(s), whether programming and/or configuring code/data. Alteration of the operational electronics typically includes some form of replacement of, or supplement to, one or more components (e.g., to provide for enhanced capabilities available by, e.g., faster circuits, more memory, multiple circuits to work in parallel, different architectures, and/or redundant circuitry).

In a typical video camera or other device of a medical imaging system, alteration of embedded software and/or operational electronics is accomplished, if at all, by providing the camera/medical device to its manufacturer or manufacturer's authorized service agent(s). This provision is achieved, e.g., by return of the camera/medical device or by in-field servicing at the site of the camera/medical device. When the camera/medical device is provided for software alteration, typically the camera/medical device is disassembled so as to replace selected, internal hardware containing the embedded software with hardware containing altered software. When the camera/medical device is provided for alteration of operational electronics, typically the camera/medical device is disassembled so as to replace selected one or more components either (i) by substituting on a chip for chip basis or, if that is not possible or reasonable due to, e.g., quality/reliability shortfalls, performance issues or chip incompatibilities (such as, divergent number or arrangement of pins, or power supply levels), (ii) by replacing one or more modules or boards (e.g., printed circuit boards), (iii) by adding new chips, modules or boards to the existing operational electronics in supplement/complement to existing operational electronics, or (iv) by some combination of the above. In some cases, manufacturers' designs may impede any such replacing, in whole or in part and, as such, may potentially impede, or even prevent, alteration of embedded software and/or operational electronics, in some ways or even entirely.

This conventional approach to alteration of medical imaging systems generally is time-consuming and potentially expensive. This is underscored especially (a) if the embedded software and/or the operational electronics tends to be altered regularly, or even with some frequency, and/or (b) if the approach contemplates the surgeon's loss of use of the camera/device while it undergoes alteration by the manufacturer.

Accordingly, a need exists for medical imaging technologies providing systems, devices and methods having field alterable electronics, e.g., which enable alteration in the field of either or both embedded software and/or operational electronics, particularly alteration that is readily accomplished by a surgeon or other end user.

### SUMMARY

The present invention satisfies this need by providing systems, devices and methods that enable alteration in the field of either or both embedded software and/or operational electronics in medical imaging technologies. Moreover, the present invention satisfies this need by so enabling alteration that is readily accomplished in the field by a surgeon or other end user.

The present invention contemplates a medical imaging system that includes a scope (e.g., an endoscope) and one or more medical devices. The system also comprises operational electronics associated with at least one of the included medical devices, embedded software associated with operational electronics so as to determine function, in whole or in part, of a medical device, and an alteration mechanism providing alteration of at least one of operational electronics and embedded software.

The present invention also contemplates a medical device that comprises operational electronics, and an alteration mechanism associated with the operational electronics, where the alteration mechanism provides alteration of operational electronics. In one aspect, the medical device has operational electronics that support a configurable component and has an alteration mechanism that provides alteration of the configurable component. In another aspect, the medical device has operational electronics that support a programmable component and has an alteration mechanism that provides alteration of the programmable component.

The present invention also contemplates a medical device that comprises operational electronics, embedded software associated with operational electronics so as to determine function, in whole or in part, of operational electronics, and an alteration mechanism, associated with the operational electronics, the alteration mechanism providing alteration of the embedded software. In one aspect, the medical device has operational electronics that support a configurable component, has embedded software that supports configuring software associated with the configurable component, and has an alteration mechanism which provides configuring alteration software that alters the configuring software. In another aspect, the medical device has operational electronics that supports a programmable component, has embedded software that supports programming software associated with the programmable component, and has an alteration mechanism which provides programming alteration software that alters the programming software.

The various features of novelty that characterize the invention are pointed out with particularity in the claims annexed to and forming a part of this specification. For a better understanding of the invention, its operating advantages and specific objects attained by its use, reference should be made to the accompanying drawings and descriptive matter in which its preferred embodiments are illustrated and described, wherein like reference numerals identify the same or similar elements.

### BRIEF DESCRIPTION OF THE DRAWINGS

FIG. 1 is a block diagram of an example embodiment of a medical imaging system, illustrating provision for alteration, according to the invention.

FIG. 2 is a block diagram of selected devices in an example embodiment of a medical imaging system, illustrating an alteration mechanism, according to the invention.

FIG. 3 is a block diagram of selected assemblies of an example embodiment of a camera in a medical imaging system, illustrating an alteration mechanism providing alteration software in the context of default software, according to the invention.

FIG. 4 is a block diagram of selected assemblies of an example embodiment of a camera in a medical imaging system, illustrating an alteration mechanism providing alteration software in the absence of default software, according to the invention.

FIG. 5 is a block diagram of selected assemblies of an example embodiment of a camera in a medical imaging system, illustrating an alteration mechanism providing alteration electronics, according to the invention.

FIG. 6 is a block diagram of selected assemblies of an example embodiment of a camera in a medical imaging system, illustrating an alteration mechanism providing interface functionality, according to the invention.

FIG. 7 is a partial, exploded, perspective view of a portion of a camera controller, including an example embodiment of an alteration mechanism, according to the invention.

### DETAILED DESCRIPTION: FIELD ALTERABLE MEDICAL IMAGING TECHNOLOGY

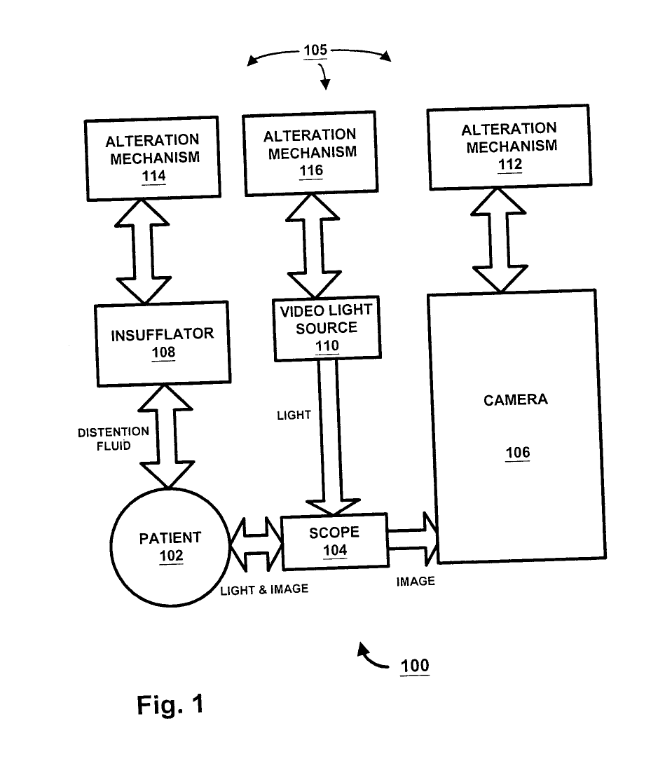

FIG. 1 is a block diagram of an example embodiment of a medical imaging system 100, illustrating provision for alteration, according to the invention. Medical imaging system 100 may be used in minimally invasive surgery so as to provide video imaging to a surgeon performing a procedure (e.g., laparoscopic surgery) on a patient 102 which procedure typically contemplates employ of surgical instruments, such as graspers, dissectors, scissors, biopsy instruments, and needle holders (not shown).

In this example embodiment, medical imaging system 100 may include one or more of the following: scope 104 and various medical devices 105. Such devices 105 may include, for example, camera 106, insufflator 108, and video light source 110. As a novel feature of the present invention, medical imaging system 100 also includes alteration mechanisms 112, 114, 116, which may be associated respectively with camera 106, insufflator 108, and/or video light source 110. Although FIG. 1 depicts alteration mechanisms 112, 114, 116 in association with each medical device 106-110 of medical imaging system 100, it is recognized that such system 100 can be implemented to associate an alteration mechanism with any one or more these or other devices, in any combination, without departing from the invention. Moreover, although FIG. 1 depicts alteration mechanisms 112, 114, 116 as completely separate from respective medical devices 106-110, it is recognized that one or more of the mechanisms 112-116 may be wholly or partly integrated with respective medical devices, in accordance with the invention.

Scope 104 typically is an optical instrument (e.g., an endoscope) which is introduced inside the patient to illuminate and acquire images of the site of the procedure. To do so, scope 104 is coupled to both video light source 110 and camera 106. Scope 104 directs to the site video light generated by video light source 110. In addition, scope 104 directs acquired images to camera 106, e.g. for observation by the surgeon in the procedure. Insufflator 108 is employed to introduce selected type(s) and amount(s) of distention fluid in association with the site to create a volume supporting performance of the procedure.

Any one or more of camera 106, insufflator 108, video light source 110 and/or other medical devices 105 of system 100 generally includes operational electronics. Operational electronics typically includes, for example, one or more control interfaces, memory systems, power supplies, printed circuit boards, and/or electrical connectors. Operational electronics' control interfaces can be variously implemented. Among other implementations, control interfaces are implemented via, e.g., control buttons, switches, lights (e.g., light emitting diodes (“LEDs”)), display panels (e.g., liquid crystal displays (“LCDs”)), speakers, microphones, keypads, touchpads, and the like. Control interfaces may be variously disposed, including, e.g., on the head board or back board of a camera controller and/or on a camera head (such controller and head are described further hereinafter). Control interfaces typically are end-user manipulatable. Control interfaces typically are applicable in receiving, processing and transmitting one or more signals, including acquired signals, feedback signals, control signals, and output signals.

Operational electronics' memory system(s) can be variously implemented. Among other implementations, a memory system is implemented to include one or more of, e.g., cache memory, dynamic random access memory (“DRAM”), static random access memory (“SRAM”), read only memory (“ROM”), electrically erasable programmable read only memory (“EEPROM”), flash memory, memory sticks, micro-drives (e.g., as provided by IBM), removable magnetic media drives (e.g., Zip drives of omega Corporation, Utah, USA, and/or conventional floppy disk drives), hard drives, and optical drives (e.g., CD-ROM, DVD-RAM), each alone or in any of various combinations. (As used herein, the term “memory system” for a device in medical imaging system 100 is used to reference any and all of the circuitry, cores, components, subsystems or systems, whether discrete, integrated in other components (e.g., as part of a microprocessor or FPGA) or otherwise, providing a memory function respecting embedded software.)

In addition to the above, operational electronics of any one or more of camera 106, insufflator 108, video light source 110 and/or other medical devices 105 of system 100 generally comprises: (a) one or more programmable components (e.g., microprocessor, microcontroller, or other similar processing unit), and/or (b) one or more configurable components (e.g., Field Programmable Gate Array (“FPGA”), Programmable Logic Device (“PLD”), Programmable Array Logic (“PAL”) and the like). More generally, operational electronics of a device 105 typically support one or more programmable components and/or one or more configurable components. Such support contemplates the alteration of operational electronics by adding one or more programmable components and/or one or more configurable components, via alteration mechanism 112, where operational electronics comprise any combination of programmable and/or configurable components prior to such alteration. In one example, such support contemplates introducing configurable components via alteration mechanism 112 where configurable components were omitted from operational electronics prior to alteration. In another example, both configurable and programmable components are introduced to the device 105, where operational electronics omit both prior to alteration.

The operational electronics of any one device of system 100 generally are implemented to respond to the function(s) associated or to be associated with that device. As an example, the operational electronics of camera 106 typically comprises one or more video sensors. Video sensors can be variously implemented, including, e.g., via charge coupled devices (“CCDs”) and/or conventional video cameras. Video sensors provide an electronic signal acquired from, and representative of, the optical images acquired and provided by scope 104. As is described in reference to other figures herein, video sensors typically are disposed in a camera head useable remotely from other parts of camera 106, but proximate to and typically in physical coupling with scope 104, which arrangement generally enhances the surgeon's facility with scope 104, e.g., in its introduction and manipulation inside patient 102.

Camera 106, insufflator 108 and video light source 110, as well as medical devices 105 generally, have associated embedded software. Embedded software, in combination with the operational electronics, generally determines the particular device's functions, features and/or performance, in whole or in part. Embedded software can determine, in whole or in part, the functions, features and/or performance of the medical imaging system 100 overall, e.g., in the coordinated operation of one or more such devices toward performing a medical procedure. (Hereinafter, the phrase “functions, features and/or performance”, and similar phrases, as well as the term comprising such phrases, are sometimes individually and collectively referred to by the term “functions”.)

It is to be recognized that other medical devices 105 may be employed in the system 100. These other medical devices 105 may be in additional to or in substitution for the above-referenced devices. In each case, as stated above, the employed medical devices 105 generally comprise embedded software and, as such, are software-determined, as described above.

Embedded software may be variously implemented. As an example, embedded software may include, without limitation, programs, directives, commands, processes, routines, information or other programming code and/or data to be executed or otherwise similarly used by a microprocessor or other programmable components (“programming software”). As another example, embedded software may include, without limitation, constants, look-up table data, information, directives, commands, routines or other configuring code and/or data to be loaded into or otherwise used to configure a FPGA or other configurable components (“configuring software”).

Embedded software can be variously embedded. As an example, embedded software can be embedded in a memory system (e.g., as firmware in a ROM). Programming software typically is so embedded. As another example, embedded software can be embedded by loading it into a device to configure one or more configurable components therein (e.g., to configure an FPGA). Configuring software typically is so embedded.

To the extent an alteration mechanism is provided in association with a respective medical device 105, the alteration mechanism may be implemented so as to enable alteration of either or both embedded software and/or operational electronics of such device 105, as determined by the device's manufacturer. In the illustrated embodiment, for example, each of alteration mechanisms 112, 114, 116 may be implemented, case by case, to enable alteration of either or both embedded software and/or operational electronics in, respectively, a camera 106, a medical device, e.g., insufflator 108, and/or a video light source 110.

Alteration of embedded software contemplates providing alteration software via respective alteration mechanism 112, 114, and/or 116. Alteration software can be provided to alter either or both programming software (“programming alteration software”) and/or configuring software (“configuring alteration software”). In either case, alteration software can alter embedded software variously, including, as examples, one or more of, in whole or in part, provision, replacement, supplement, update, upgrade, debugging, compaction, or any other addition or change relating to code, data and/or other software aspects, with or without improvement(s). It is to be recognized that alteration software can alter embedded software when embedded software is omitted, e.g. introducing software to overcome the omission. In that case, embedded software is understood to support programming software and/or configuring software, including, during alteration, by introducing either or both when such software is omitted prior to alteration. (Herein, the terms “alteration”, “alter”, “altering” and the like, when applied to software, have any and all of the specific meanings set forth above, as well as the meanings generally referenced.)

Alteration software is implemented in the context of the embedded software. In one example, the medical device 1 05 has embedded software that is used when some or all of the alteration software is not used (“default software”). In this example, the alteration can be (a) temporary, e.g., continuing only while an alteration mechanism is present, triggered, selected or otherwise operational, or (b) permanent, e.g., by overwriting the default software or by otherwise destroying default software. In the temporary case, the alteration mechanism may be triggered, selected or otherwise operational with or without the user's action (e.g., triggered by the user's input via control interfaces; e.g., triggered by the user's coupling of a specific scope or camera head or other medical device in or to the system 100; e.g., automatically by the system 100 detecting the alteration mechanism or alteration software).

In another example, alteration software is provided via an alteration mechanism 112, 114, 116 associated with the respective device where the device has no default software. This example generally contemplates that the respective device 105 is inoperable, except for an alteration mechanism 112, 114, 116 providing the alteration software. This inoperability can range in effect, e.g., it can be either in part (e.g., as to the functions associated with the alteration software) or complete (i.e., the device is inoperable as to all functions, features and performance). Moreover, when the alteration software is provided by the alteration mechanism, the alteration can be: (a) temporary, e.g., continuing only if and while an alteration mechanism is present, triggered, selected or otherwise operational, (b) quasi-permanent, e.g., by reading alteration software from respective alteration mechanism 112, 114, 116 for writing into a respective device so as to enable that software to be present and have effect, but with exposure to over-writing, or (c) permanent, e.g., by reading alteration software from alteration mechanism 112, 114, 116 for writing into a respective device so as to enable that software to be present and have effect, while proscribing over-writing.

Alteration of embedded software can provide various results. Generally, alteration can change, e.g., individual functions, in whole or in part, of one or more medical devices 105 and can change overall functions, in whole or in part, of medical imaging system 100. Some changes include, e.g., enhanced reliability, faster operation, enhanced personalization, new functions, new features, improved functions, improved features, and the like.

Alteration of the operational electronics can be variously implemented. As general examples, alteration of operational electronics contemplates any of, in whole or in part: addition, replacement, or supplement/complement of/to one or more circuits, components, modules, boards, subsystems, systems, or some combination. Within these general examples, alteration of operational electronics may contemplate actually or effectively replacing existing operational electronics, in whole or in part (e.g., effective replacement being where the existing operational electronics remain, but have functions taken over by alteration electronics, as described below). Yet also within the above-referenced general examples, alteration of operational electronics may contemplate supplementing/complementing existing operational electronics, in whole or in part (e.g., to provide functionality of which the existing operational electronics is not capable or not sufficiently capable). Still also within the above-referenced general examples, alteration of operational electronics may contemplate adding operational electronics where none existed (e.g., some or all of operational electronics is provided only via an alteration mechanism 112, 114, 116, such that the respective device of the system 100 is inoperable, in whole or in part unless the alteration mechanism 112, 114, 116 so provides the electronics). In such case, the alteration mechanism works to overcome the omission of some or all of the operational electronics, in whole or in part. Covered by this lattermost case is the addition of interface functionality not provided by operational electronics (e.g., by providing inpuvoutput ports, such as one or more serial port, parallel port, system bus, universal serial bus ports, IEEE 1394 interface ports (“firewire”), ethernet interfaces, and/or other network interfaces). As described further herein, it is recognized that alteration of operational electronics (e.g., to add interface functionality or to overcome other omission of electronics) typically is coordinated with alteration of embedded software (e.g., when embedded software is omitted, by introducing software to overcome the omission). (Herein, the terms “alteration”, “alter”, “altering” and the like, when applied to operational electronics, have any and all of the specific meanings set forth above, as well as the meanings generally referenced.)

Alteration of operational electronics is achieved in a device of system 100 by providing alteration electronics via a respective alteration mechanism 112, 114, 116. In doing so, various results may be provided. Generally, alteration of operational electronics can change, e.g., individual functions, in whole or in part, of one or more medical devices 105 and can change overall functions, in whole or in part, of medical imaging system 100. Some examples of changes include enhanced reliability, faster operation, enhanced personalization, new functions, new features, improved functions, improved features, and the like, such as through, e.g., faster circuits, more reliable circuits, an expanded or otherwise enhanced memory system, multiple circuits working in parallel, redundant circuitry, and/or new architectures.

Alteration of operational electronics preferably is realized through appropriate coordination with alteration of embedded software. Such coordination enables, e.g., mutual support of alterations and avoidance of conflicts, generally. Such coordination can include, for example, provision of configuring alteration software in association with alteration electronics that are configurable (“configurable alteration electronics”). In such example, all or part of such configuring alteration software is loaded with and configures, in whole or in part, such configurable alteration electronics. To illustrate such example, alteration mechanism 112 can be implemented as a circuit board (“alteration board”) which is electrically and removably introducable into a medical device 105, which comprises an FPGA, and which provides configuring alteration software that is (i) present on the alteration board in an EEPROM or otherwise from memory system, for loading and configuring such FPGA, in whole or in part, or (ii) already loaded in and configuring such FPGA (i.e., the alteration board is marketed with the FPGA configured, in whole or in part by configuring alteration software), or (iii) loaded via a network connection provided by the alteration board, or (iv) loaded from or through device 105, such as by having a portion of alteration mechanism 112 separate from the alteration board (e.g., an I/O connection or other network connection) or by using the device's memory system to store previously-provided configuring alteration software, or (v) provided via some combination of the above.

It is contemplated that one or more configurable alteration electronics may be implemented so as to be configured by other than configuring alteration software. In such case, such components may be configured, in whole or in part, using or based on embedded software of the device 105.

It is also contemplated that, if configurable alteration electronics are provided in association with a device comprising configurable components, configuring alteration software may be provided which configures, in whole or in part, either configurable alteration electronics, or configurable components, or both such components.

It is also contemplated that configurable alteration electronics are available loaded and configured at some predetermined time or upon some predetermined event. As an example, such loading/configuring can be at first start up of device 105 after alteration electronics are introduced. As another example., such loading/configuring can be at manufacture.

In another example, such coordination can include provision of programming alteration software in association with alteration electronics that are programmable (“programmable alteration electronics”). In such example, all or part of such programming alteration software is available to and programs, in whole or in part, such programmable alteration electronics. To illustrate this example, alteration mechanism 112 can be implemented as the alteration board described above to comprise configurable alteration electronics, where the alteration board further comprises a microprocessor and provides for programming alteration software which is provided (i) on the alteration board in an EEPROM or otherwise from memory system, or (ii) via a network connection of the alteration board, or (iii) from or through device 105, such as by having a portion of alteration mechanism 112 separate from the alteration board (e.g., an I/O connection or other network connection) or by using the device's memory system to store previously-provided programming alteration software, or (iv) provided via some combination of the above.

It is contemplated that one or more programmable alteration electronics may be implemented so as to omit, or to be programmed by other than, programming alteration software. In such case, the components may have available, and be programmed, in whole or in part, using or based on, embedded software of the device 105.

It is also contemplated that, if programmable alteration electronics are provided in association with a device comprising programmable components, programming alteration software may be provided which programs, in whole or in part, either programmable alteration electronics, or programmable components, or both such components.

It is also contemplated that programmable alteration electronics have access to programming alteration software and/or embedded software at some predetermined time or upon some predetermined event, e.g., at first start up of device 105 after alteration electronics are introduced or at manufacture.

Notwithstanding the examples of coordination described above, it is recognized that alteration of operational electronics may be performed without alteration of embedded software associated with altered operational electronics and, as well, without alteration of any embedded software. Typically, in such case, embedded software executes on or in connection with alteration electronics.

To illustrate this case, alteration mechanism 112 can be implemented as an alteration board which is electrically and removably introducable into a medical device 105, which comprises configurable and/or programmable alteration electronics, and/or configuring and/or programming alteration software, so as to support interface functionality comprising networking. To do so, such alteration electronics generally includes an appropriate network connector and such alteration software generally includes a communication stack (e.g., a TCP/IP stack). In an example embodiment of this case, the alteration electronics and software enables selected functionality via the network, such as, as an example, alteration of embedded software (e.g., automatic detection or manual entry of the configuration of system 100 so as to download current versions of some or all of embedded software, or selected embedded software, where this download may be subject to authentication and other security limitations (i.e., passwords, biometric identification, etc.) as well as various commercial payment terms). Wireless communication protocols and applicable hardware are also contemplated in the foregoing regards.

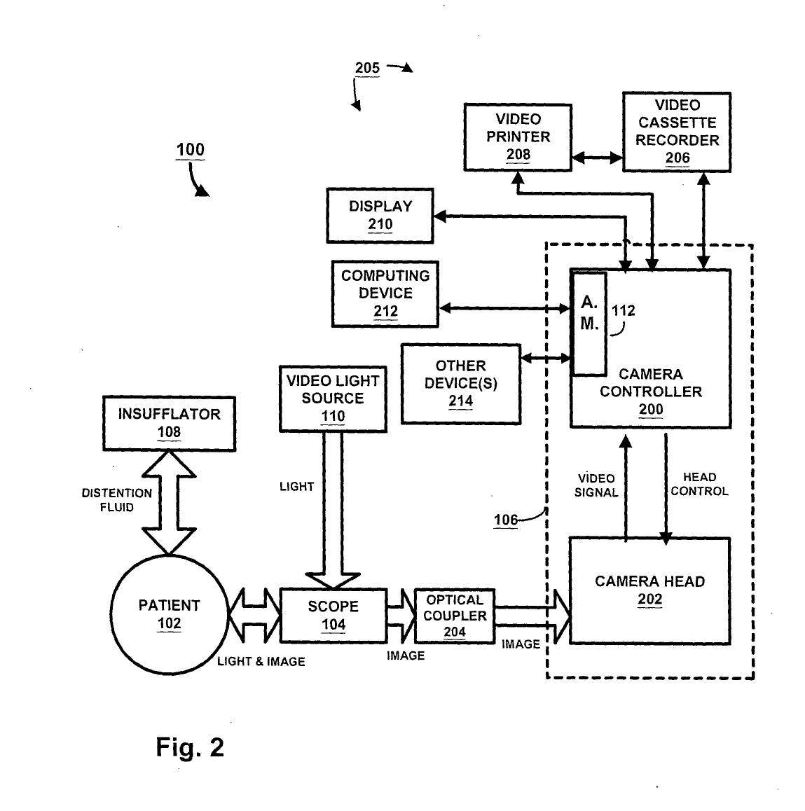

FIG. 2 is a block diagram of an example embodiment of medical imaging system 100. It is noted that variations of the features of system 100 are possible, and FIG. 2 is for illustrative purposes only. In this example, camera 106 has an associated alteration mechanism 112. At the same time, camera 106 is illustrated to comprise camera controller 200 and camera head 202. Camera head 202 is coupled to scope 104 via optical coupler 204 so as to receive optical images of the site inside patient 102. Camera head generates one or more video signals representative of the optical images, providing such signal(s) to camera controller 200.

Camera controller 200 provides head control signals to camera head 202. The head control signals include, e.g., shutter speed controls. Shutter speed controls for a CCD determine the time period over which charge is accumulated for the pixels of each field/frame of the video signal (e.g., greater time periods of accumulation generally result in relatively more accumulated charge and, in turn, greater intensity of the video image associated with the video signal arising from such charge). Shutter speed is typically based, in part, on digital signal processing of the video signal itself.

Camera controller 200 and head 202 may comprise a camera in a single unit. However, as indicated here, camera head 202 may be implemented so as to be coupled to (e.g., wired or wireless) camera controller 200. In such case, head 202 and controller 200 are implemented other than in a monolithic, single unit, so that camera head 202 is enabled for use remotely from camera controller 200, but still under electronic control of controller 200. Moreover, camera head 202 is enabled for use proximate to, and typically in physical coupling with, scope 104, which arrangement generally enhances the surgeon's facility with scope 104, e.g., in its introduction and manipulation inside patient 102.

Camera controller 200 supports one of more input/output devices 205. These include, as examples, videocassette recorder 206, video printer 208, display 210, computing device 212, and one or more other device(s) 214. Each input/output device 205 may be coupled to controller 200, such coupling being, e.g., via wire, cable, radio signal, infrared signal or other mechanical or electromagnetic solution, using any protocol (e.g., USB, IEEE 1394, PCI, TCP/IP, etc.). Moreover, any or all of input/output devices 206-214 may be coupled to any one or more other of input/output devices 205. For example, as shown, video printer 208 is also coupled to video cassette recorder 206, indicating that printing may directed from recorder 206 directly to printer 208, separately from operation of camera 106.

Display 210 may be variously implemented. As examples, display 210 may be implemented to comprise a video monitor and/or head mounted display.

Computing device 212 may be variously implemented. As examples, computing device 212 may be implemented to comprise: a personal computer; a network appliance; a personal digital assistant (e.g., a Palm VIIx); a server linked to the Internet, an intranet or other network; other similar devices; and/or combinations of any of these. In the server case, the server could be used to provide access to video of the procedure, either openly or under secure conditions, and either/both real-time or/and after the completion of the procedure.

Other device(s) 214 may be variously implemented. As examples, device(s) 214 can be implemented as an optical recorder (e.g., CD or DVD), or as a broadcast mechanism providing for various forms of broadcast of the video signal (e.g., throughout an operating room), or to establish a network link. In the latter case, device 214 may comprise a router, switch, bridge, gateway and/or any other network component, or combination of components, appropriate to networking, as well as supporting tasks. A network link can be implemented, as an example, to provide for distribution of video to remote locations, such as (a) for training of other surgeons or medical students in the procedure captured in the video and/or (b) for enabling the procedure to be performed by a surgeon remote from patient 102 and system 100. In so enabling a remotely-located surgeon, the network link is contemplated to be implemented both to transmit video and to receive control instructions (e.g., directing the operation of camera 106, enabling controlled movement of both scope 104 and other instruments, and otherwise enabling operation of the system 100 for and during the procedure).

In the example embodiment of FIG. 2, alteration mechanism 112 is associated with controller 200. In particular, alteration mechanism 112 is depicted as providing interface functionality. More specifically, the instant alteration electronics (not shown) provided via alteration mechanism 112 are depicted to support input/output ports, such as one or more serial ports, parallel ports, USB ports, firewire ports, system bus connectors, ethernet interfaces, and/or other network interfaces. The input/output ports enable coupling of controller 200 to the devices 212, 214. Indeed, these ports could be provided (in another example embodiment) via the alteration mechanism 112 to provide all interface functionality for controller 112, so as to enable coupling of controller 200 not only to computing device 212 and other device(s) 214, but also to recorder 206, printer 208 and display 210.

It is to be recognized that, although alteration mechanism 112 is depicted within the block representing camera controller 200, that depiction is for illustrative purposes. In this example embodiment, mechanism 112 may be implemented for, e.g., removable insertion within controller 200. However, mechanism 112 may also be otherwise and variously implemented, such as, e.g., external to controller 200 and linking thereto through one or more connections supported by controller 200. Whatever the implementation, alteration mechanism 112 is implemented so as to provide for alteration of embedded software and/or operational electronics of one or more devices of medical imaging system 100, by providing either/both alteration software and/or alteration electronics. As another example, communications port may be provided in the medical device for communicating with a remote alteration mechanism over a wireless connection.

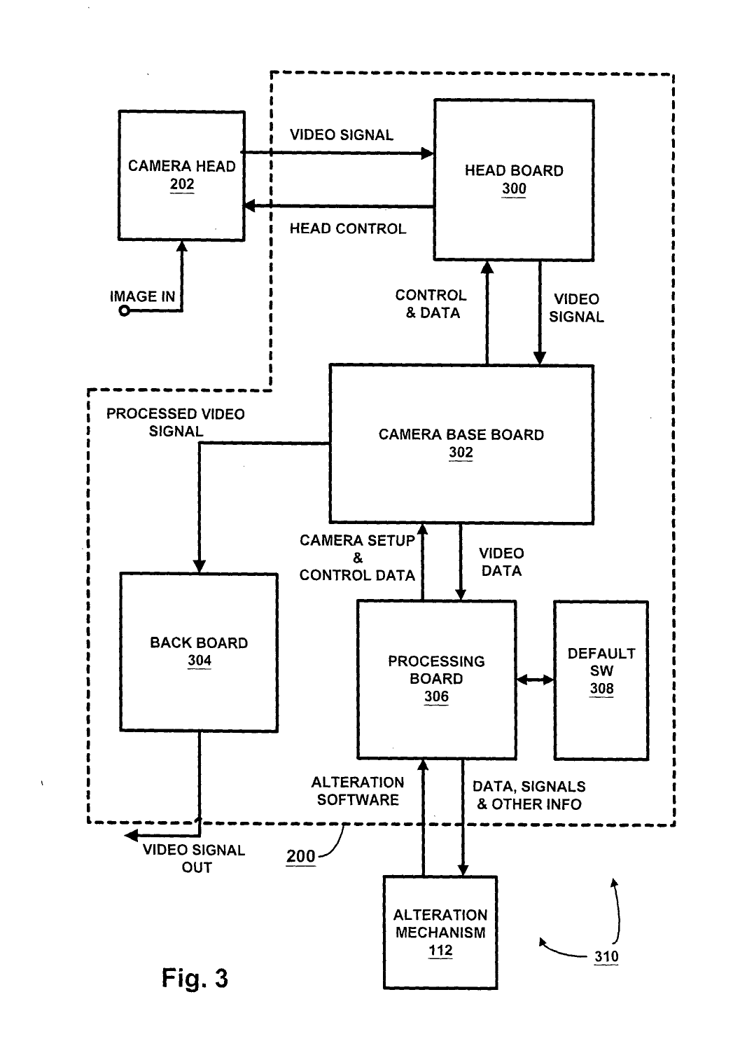

FIG. 3 is a block diagram of an example embodiment of camera 106 comprising camera controller 200 and camera head 202 in medical imaging system 100. Camera controller 200 generally comprises one or more assemblies 310. In this example embodiment, camera controller 200 is depicted to comprise assemblies 310 that include head board 300, camera base board 302, back board 304, and processing board 306. Camera controller 200 has associated therewith default software 308 and, for provision of alteration software, alteration mechanism 112. (Provision of alteration electronics, with and/or without alteration software directed to existing operational electronics of system 100, via alteration mechanism 112, is also contemplated. Examples of provision of alteration electronics are described hereinafter, with reference to additional figures.)

As previously described with reference to other example embodiments, camera head 202 is attached, e.g., to scope 104, so as to receive optical images, e.g., of the site of a procedure. Camera head 202 generally also comprises a CCD and/or one or more other video sensors, as well as lenses, focus rings and a shutter control device, so as to enable camera head 202 to generate a video signal representative of the received optical images.

Camera head 202 is coupled to head board 300 of camera controller 200. As such, camera controller 200 provides head control signals to camera head 202, and receives the video signal generated by camera head 202. It is contemplated in this embodiment that camera head 202 also receives control and/or data signals from the processing board 306 (e.g., including one or more such signals being passed through—substantially or exactly—base board 302 and head board 300). Such received control signals typically include shutter control signals for camera head 202. As earlier noted, the camera and head need not be separate components; they may be implemented as a single monolithic structure, in a miniaturized form factor, and in other ways that will be appreciated by persons skilled in the art from the teachings herein.

Head board 300 typically provides control interfaces. As previously described, control interfaces can be variously implemented, including, e.g., via control buttons, switches, LEDs, LCDs, speakers, microphones, keypads, touchpads, and the like. Control interfaces typically are end-user manipulatable in association with, to trigger, to control or otherwise to support one or more features or functions of camera 106, controller 200 or system 100. Control interfaces typically are applicable in receiving, processing and transmitting one or more signals, including one or more of acquired signals, feedback signals, control signals, data signals and output signals. It is contemplated in this embodiment that the control interfaces would be manipulatable, e.g., to manually trigger a white balance, as well as to set gain/picture level.

Camera base board 302 provides a processed video signal to back board 304. Back board 304 provides for translation, if any, of the processed video signal to an output signal appropriate, e.g., to drive display 210. Back board 304 typically provides interface functionality, e.g., input/output ports, such as RGB and/or composite video. Back board 304 may also be implemented to provide control interfaces, e.g., directed to interface functionality.

Processing board 306 is associated with camera base board 302 and, through base board 302, with back board 304, head board 300 and camera head 202. Processing board 306 may be variously implemented. Generally, processing board 306 comprises operational electronics, including either or both programmable components or/and configurable components. In an example embodiment, processing board 306 comprises both a microprocessor (i.e., a programmable component) and an FPGA (i.e., a configurable component). In such example embodiment, the processing board 306 typically includes all or part of the controller's memory system. The processing board's memory system may be implemented to reside in one or more of (i) the microprocessor (e.g., on board cache), (ii) the FPGA and/or (iii) other circuitry. Such memory system generally contains embedded software of the controller, in whole or in part.

Processing of the video signal (e.g., white balance, gain/picture level adjustment and other video signal processing) typically is performed by processing board 306. Video signal processing generally is performed via processing board's operational electronics operating responsive to embedded software: e.g., programmable components operating responsive to programming software and/or configurable components operating responsive to configuring software. In such processing, the embedded software may be implemented to take into account one or more various factors, including, e.g., the nature and location of the procedure, the performance parameters of scope 104, the performance parameters of camera head 202, the performance parameters of other associated devices (e.g., insufflator 108, light source 110, recorder 206, printer 208, display 210, computing device 212 and/or other devices 214), and/or the type and nature of the hand instruments to be used in the procedure.

Video signal processing may also be performed by processing board 306 operating in conjunction with camera base board 302. In such case, operational electronics of the camera base board 302 typically operate responsive to embedded software. As an example, a particular base board 302 may include registers, electronically variable resistors (EVRs which can function as registers) or the like, such that levels may be written to such registers/EVRs for, e.g., establishing shutter speeds and/or picture levels. In such case, depending on the operation being performed, processing board 306 may (i) perform in conjunction with base board 302, (ii) perform alone (i.e., base board 302 does not contribute in video signal processing), or (iii) not perform video signal processing (i.e., base board 302 performs the operation alone).

In this example, embedded software is provided, in whole or in part, by default software 308. If alteration mechanism 112 is not present, triggered, selected or otherwise operational or is not directed to an operation, default software 308 provides for such operation. If alteration mechanism 112 is present and is directed to an operation, alteration mechanism 112 has alteration software that alters the default software 308.

Such alteration can have various effects. As an example, such alteration can be temporary; e.g., continuing only while alteration mechanism 112 is present, triggered, selected or otherwise operational. As another example, such alteration can be permanent, e.g., by overwriting some or all of the default software 308 or by otherwise destroying default software 308. As yet another example, such alteration can be a combination of both temporary and permanent, e.g., feature by feature, function by function, configuring vs. programming software, etc.

In the case of temporary alteration, default software 308 preferably is provided in a fixed form. Examples of fixed form include, for programming software, a ROM and, for configuring software, a gate array or other, non-field-configurable logic. In the example of default software configuring a gate array, alteration mechanism 112 may be implemented to introduce a second logic device to perform preferentially one or more of the functions provided by that gate array. In such case, a device supporting alteration mechanism 112, e.g., camera 106 as depicted in FIG. 3, may be implemented so as to detect when the mechanism 112 is present, triggered, selected or otherwise operational, so that operational control is transferred from the gate array (i.e., default software 308) to the appropriate circuitry (e.g., address) of the gate array 112, or otherwise to the appropriate alteration software.

In the case of permanent alteration, default software 308 preferably is provided so as to enable it being overwritten, in whole or in part, as the case may be, with alteration software from alteration mechanism 112. Examples include, for programming software, provision of default software in static or dynamic RAM, EEPROMs and other writable components of memory system. Examples also include, for configuring software, provision of default software in FPGA, PLD and other field-configurable logic.

In the case of permanent alteration, a device supporting alteration mechanism 112, e.g., camera 106 as depicted in FIG. 3, may be implemented so as to detect when the mechanism 112 is present, triggered, selected or otherwise operational, so that (a) programming alteration software over-writes appropriate parts of the programming software portion of default software 308 (e.g., at appropriate addresses in the memory system of camera 106) and/or (b) configuring alteration software is loaded into and configures applicable configurable components, thereby effectively overwriting appropriate parts of the configuring software portion of default software 308. In doing so, operational control may be properly transferred from default software 308 to the appropriate parts (e.g., circuit and/or address) of alteration mechanism 112, or otherwise to the appropriate alteration software, as the corresponding parts of default software 308 are destroyed.

Whether temporary, permanent or combination alteration is implemented, the device 105 associated with alteration mechanism 112 in system 100 preferably provides for detection of the mechanism's presence, trigger, selection or other operation. In the depicted embodiment of FIG. 3, this detection preferably is performed by any one or more of the assemblies of camera controller 200 which exploit the alteration mechanism, e.g., processing board 306 and/or camera base board 302. The detection can be variously initiated (e.g., initiated at power up).

Alteration can be in whole or in part. As an example, alteration can be established as always total or always of certain portions of default software 308. As another example, alteration can be implemented using one or more flags or other indicators which, when of one or more states, directs that alteration is total and, when of another or other state(s), directs that alteration is partial. In the case of partial alteration, the flags or other indicators, or some other setting, preferably identifies the portion(s) of default software 308 to be altered.

Any alteration may implemented subject to certain restrictions. Examples of restrictions include, without limitation, passwords, electronic signatures, fees, and hardware and/or software configuration requirements.

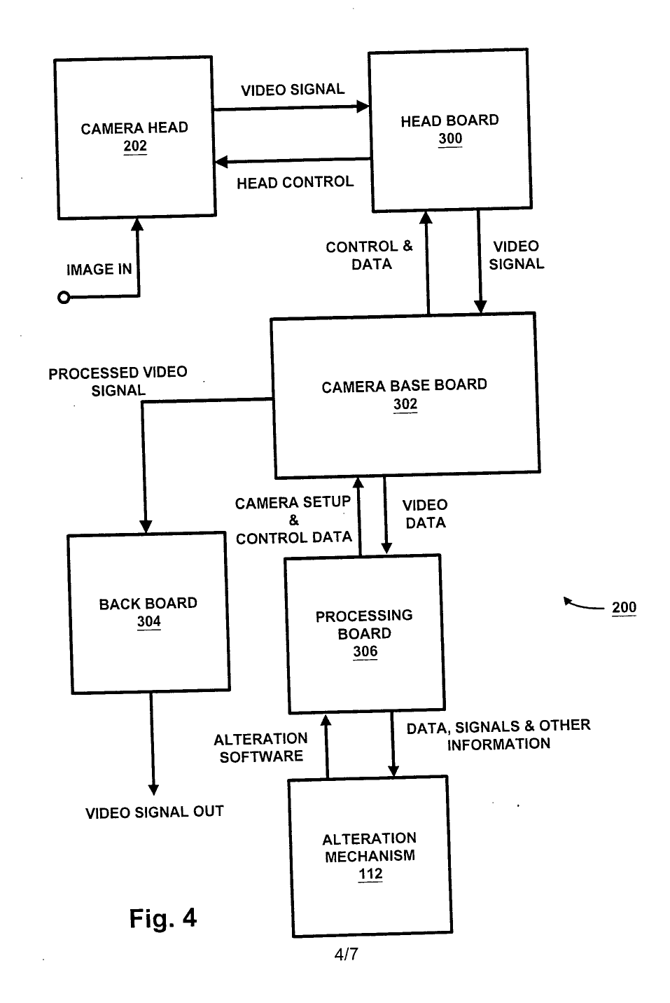

FIG. 4 is a block diagram of an example embodiment of camera 106 comprising camera controller 200 and camera head 202, in medical imaging system 100. Camera controller 200, in this example embodiment, is depicted to comprise various, previously-described assemblies 310. Moreover, as also previously described, camera controller 200 has associated therewith alteration mechanism 112, for provision of alteration software.

However, by comparison to the example embodiment depicted in FIG. 3, camera controller 200 omits default software 308. Because the default software 308 is omitted, camera 106 is inoperable as to any operations that the alteration software supports, unless and until alteration mechanism 112 is present, triggered, selected or otherwise operational. In such case, the implicated operations of camera 106 are alterable depending on the implementation of alteration software. Moreover, because alteration software generally may be changed from time to time, the implicated operations can be altered from time to time. As an example, alteration mechanism 112 may provide for swapping alteration boards, particularly to select performance specific to the alteration software of a selected board (use of circuit boards in implementing alteration mechanism 112 is discussed elsewhere herein).

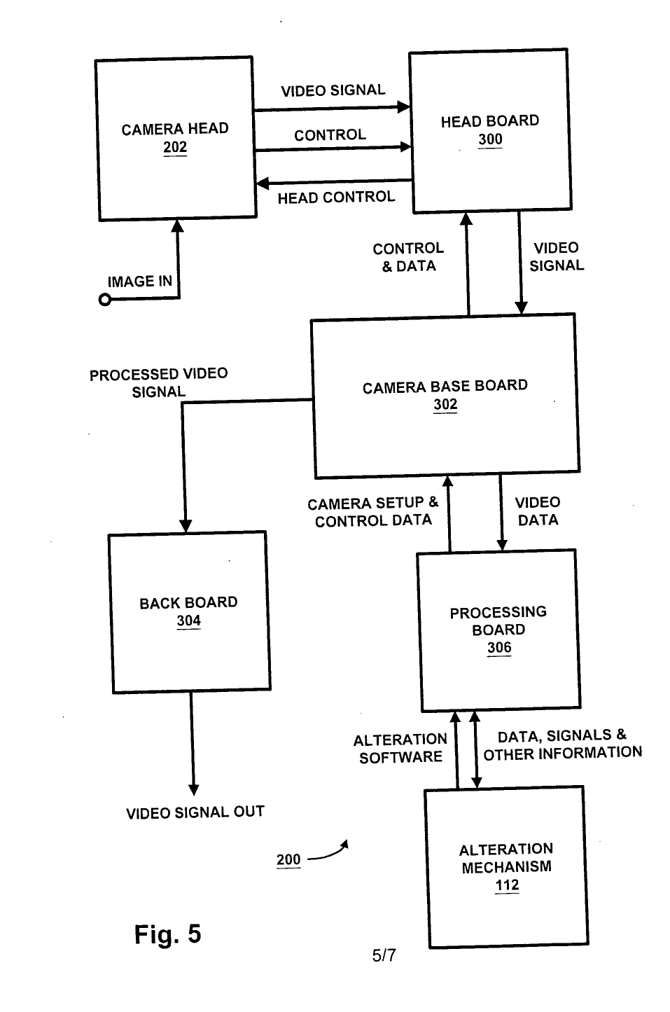

FIG. 5 is a block diagram of an example embodiment of camera 106 comprising camera controller 200 and camera head 202, in medical imaging system 100. Camera controller 200, in this example embodiment, is depicted to comprise various, previously-described assemblies 310 and has associated therewith alteration mechanism 112 for provision of alteration software. FIG. 5's camera controller 200 omits default software 308 and, accordingly, has characteristics as described for such omission with reference to FIG. 4. Moreover, FIG. 5's alteration mechanism 112 is depicted not only to receive, but also to provide data, signals and other information to processing board 306 of controller 200. This provision indicates that alteration mechanism 112 supports alteration electronics.

The camera head 202 of FIG. 5 is depicted to provide control signals to camera controller 200 and, in particular, to head board 300. These control signals indicate that camera head 202 provides control interfaces, as previously described (e.g., typically user manipulatable control interfaces). In this example embodiment, control interfaces may be disposed at the camera head 202 to advance the surgeon's facility in exploiting some or all features, functions and performance of camera 106, via camera head 202, remote from controller 200 and, thereby, advancing the surgeon's efficiency and productivity in medical procedures.

The control interfaces associated with camera head 202 may be implemented to support functions provided by operational electronics of camera controller 200. It is also contemplated that such control interfaces may be implemented to support functions provided by either or both alteration software and/or alteration electronics provided by alteration mechanism 112, alone or in combination with operational electronics of camera controller 200.

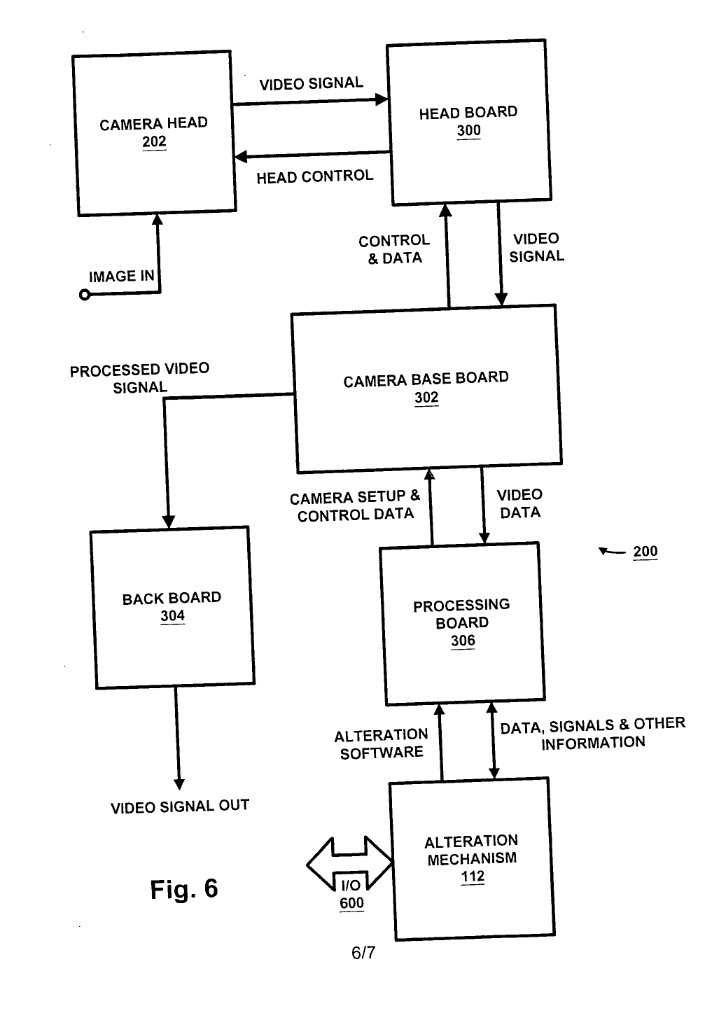

FIG. 6 is a block diagram of an example embodiment of camera 106 comprising camera controller 200 and camera head 202, in medical imaging system 100. Camera controller 200, in this example embodiment, is depicted to comprise various, previously-described assemblies 310 and has associated therewith alteration mechanism 112 for provision of alteration software. FIG. 6's camera controller 200 omits default software 308 and, accordingly, has characteristics as described for such omission with reference to FIG. 4.

FIG. 6's alteration mechanism 112 is also depicted to provide data, signals and other information to processing board 306 of controller 200. This provision indicates that alteration mechanism 112 supports interface functionality, represented by I/O 600. This interface functionality may be implemented for support by operational electronics of camera controller 200. It is also contemplated that this interface functionality may be implemented for support via alteration mechanism 112, e.g., wherein the functions respecting such interface functionality are supported by either or both alteration software and/or alteration electronics provided by alteration mechanism 112. It is also contemplated that this interface functionality may be implemented for support by a combination of operational electronics of camera controller 200, and/or alteration software of alteration mechanism 112, and/or alteration electronics of such mechanism 112.

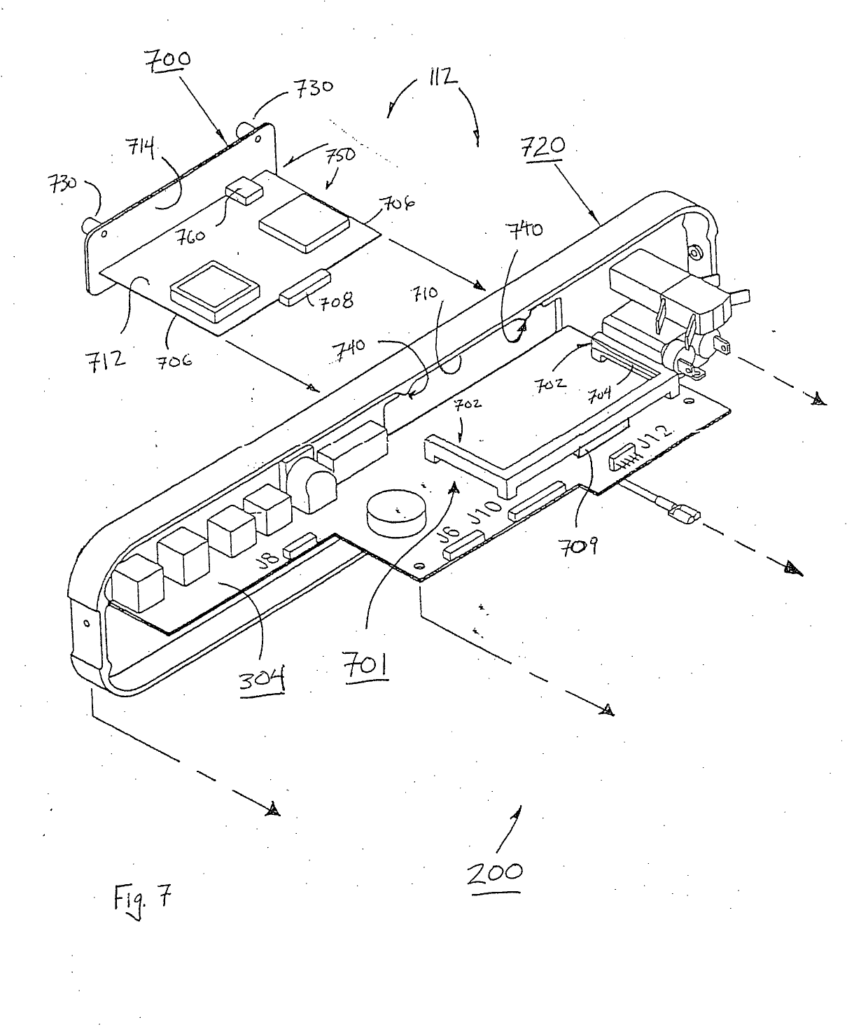

FIG. 7 is a partial, exploded, perspective view of an example embodiment of camera controller 200 of medical imaging system 100, including provision for alteration mechanism 112. In this example embodiment, alteration mechanism 112 comprises alteration board 700 and receiving mechanism 701. Alteration board 700 comprises rails 706 and first electrical connector 708. Receiving mechanism 701 comprises insertion guides 702, slots 704 and second electrical connector 709. Receiving mechanism 701 of this embodiment of alteration mechanism 112 is mounted on back board 304 of controller 200. It is to be recognized that receiving mechanism 701 may be otherwise associated with camera controller 200, including, as an example, by being mounted on processor board 306.

As illustrated in this example embodiment, alteration board 700 is removably insertable into receiving mechanism 701. More specifically, alteration board 700 is removably insertable by (a) introducing the board 700 through opening 710 disposed in back panel 720 (back panel 720 here carries back board 304 of controller 200), (b) inserting rails 706 into slots 704 via insertion guides 702 (e.g., insertion guides are here depicted as being flared so as to enhance properly aligned insertion), and (c) electrically engaging first electrical connector 708 of the board 700 with second electrical connector 709 of the receiving mechanism 701.

Second electrical connector 709 preferably is electrically connected, in turn, with one or more applicable assemblies 310 of camera controller 200. As an example, connector 709 may be electrically connected in this example embodiment to either/both processing board 306 and/or camera base board 302. Moreover, connector 709, while illustrated here as an integral part of receiving mechanism 701, may be otherwise implemented, including, as examples, mounted on/to one of the controller's other assemblies 310 (e.g., back board 304, processing board 306 or camera base board 302).

In this example embodiment, alteration board 700 comprises printed circuit board 712 which is coupled with and to plate 714. Plate 714 preferably is constructed of material(s) the same or similar to that used for the controller's box (e.g., aluminum or other metal, or plastic). While a printed circuit board is depicted, it is recognized that other component(s) can be used in place of such board, provided such component(s) enable the introduction of alteration electronics and/or alteration software (e.g., a smart card, or PC card).

Once inserted in receiving mechanism 701, alteration mechanism 112 preferably enables alteration board 700 to be removably securable in place. Alteration mechanism 112 can be variously implemented to do so. As illustrated, alteration board 700 is securable via one or more (e.g., 2) thumbscrews 730 disposed in plate 714 which the user can screw into respective receptacles 740, which receptacles 740 are either integral with or mechanical attached to back panel 720. With thumb screws 730 adequately screwed into respective receptacles 740, alteration board 700 is secured in place, via plate 714, to back panel 720 of camera controller 200. However, alteration board 700 remains removable merely by unscrewing thumb screws 730.

While thumbscrews 730 are illustrated, it is understood that alteration board 700 is securable in place via technologies and solutions other than thumbscrews 730. Example include, without limitation, set screws, clips, and/or a locking mechanism.

Alteration board 700, as illustrated, comprises electronics 750. In an example embodiment, electronics 750 comprises one or more EPROMs (Erasable Programmable Read Only Memory), EEPROMS (Electrically Erasable Programmable Read Only Memory) or other memory devices (such as, without limitation, smart cards, flash memory cards, memory sticks, replaceable micro-drives and the like) of a memory system. In operation, the memory devices may contain programming software to be executed on the programmable components of camera controller's operational electronics (by any of various approaches described herein). Further, the memory devices may contain configuring software for loading into and configuring configurable components of the camera controller's operational electronics. The memory devices may also contain both programming and configuring software in which case electronics 750 may be implemented (i) with either one memory device for both types of software and/or (ii) with a separate memory device for each of the types of software.

In another example embodiment, electronics 750 comprises alteration electronics. Such alteration electronics can be implemented to include, as previously described, programmable alteration electronics, configurable alteration electronics, or a combination. In one case of such example embodiment, electronics 750 implements interface functionality, e.g., I/O ports.

In yet another example, electronics 750 comprises elements that provide a desired combination of alteration electronics and alteration software.

In addition to medical imaging applications, the present invention may also be used in other applications of imaging systems. For example, industrial endoscopes are used in the inspection of machines and other items with hard to reach places. The endoscopes may be used in conjunction with working devices, such as a camera and/or micro-tools to repair or replace components of machinery.

The foregoing embodiments and features are for illustrative purposes. Persons of ordinary skill in the art will recognize the foregoing description and embodiments are not limitations, but examples. Such persons will recognize, in particular, that many modifications and variations are possible in the details, materials, and arrangements of the parts and steps which have been described and illustrated in order to explain the nature of this invention, and that such modifications and variations do not depart from the spirit and scope of the teachings and claims contained herein.

1. A medical device, comprising:
operational electronics; and
an alteration mechanism associated with the operational electronics, the alteration mechanism providing alteration of operational electronics.

2. The medical device of claim 1, wherein the operational electronics supports a configurable component and the alteration mechanism provides alteration of the configurable component.

3. The medical device of claim 2, wherein the operational electronics supports a programmable component and the alteration mechanism provides alteration of the programmable component.

4. The medical device of claim 2, wherein the configurable component supports configuring software and the alteration mechanism comprises configuring alteration software that alters the configurable component by altering the configuring software.

5. The medical device of claim 4, wherein alteration of the configurable component is temporary.

6. The medical device of claim 4, wherein configuring software is omitted, in whole or in part, and the configuring alteration software alters the configurable component by overcoming omission of the configuring software, in whole or in part.

7. The medical device of claim 4, wherein the alteration mechanism comprises alteration electronics, the alteration electronics providing alteration of the configurable component.

8. The medical device of claim 7, wherein the configurable component is omitted from the operational electronics, in whole or in part, and the alteration electronics provides alteration by overcoming omission of the configurable component, in whole or in part.

9. The medical device of claim 2, wherein the alteration mechanism comprises alteration electronics, the alteration electronics providing alteration of the configurable component.

10. The medical device of claim 9, wherein the configurable component is omitted from the operational electronics, in whole or in part, and the alteration mechanism provides alteration electronics which alter the operational electronics by overcoming omission of the configurable component, in whole or in part.

11. The medical device of claim 10, wherein the operational electronics supports a programmable component and the alteration electronics alter the programmable component.

12. The medical device of claim 11, wherein the programmable component is omitted from the operational electronics, in whole or in part, and the alteration mechanism provides alteration electronics which alter the operational electronics by overcoming omission of the programmable component, in whole or in part.

13. The medical device of claim 9, wherein the operational electronics supports a programmable component and the alteration electronics alter the programmable component.

14. A medical device, comprising:
operational electronics; and
an alteration mechanism associated with the operational electronics, the alteration mechanism providing alteration of the operational electronics, such alteration including provision of interface functionality.

15. The medical device of claim 14, wherein the alteration mechanism comprises an alteration board.

16. The medical device of claim 14, wherein the alteration mechanism supports networking.

17. A medical device, comprising:
operational electronics;
embedded software associated with operational electronics so as to determine function, in whole or in part, of operational electronics; and,
an alteration mechanism, associated with the operational electronics, the alteration mechanism providing alteration of the embedded software.

18. The medical device of claim 17, wherein the operational electronics supports a configurable component, the embedded software supports configuring software associated with the configurable component, and the alteration mechanism provides configuring alteration software that alters the configuring software.

19. The medical device of claim 18, wherein the operational electronics supports a programmable component, the embedded software supports programming software associated with the programmable component, and the alteration mechanism provides programming alteration software that alters the programming software.

20. The medical device of claim 19, wherein the programming software is omitted, in whole or in part, and the programming alteration software alters the embedded software by overcoming omission of the programming software, in whole or in part.

21. The medical device of claim 18, wherein the configuring software is omitted, in whole or in part, and the configuring alteration software alters the embedded software by overcoming omission of the configuring software, in whole or in part.

22. The medical device of claim 17, wherein the embedded software includes default software and the alteration mechanism provides alteration software, such that the default software determines function, in whole or in part, of the operational electronics when the alteration software is not used.

23. The medical device of claim 17, wherein the alteration software provides alteration that is temporary.

24. The medical device of claim 17, wherein the alteration software provides alteration that is quasi-permanent.

25. The medical device of claim 17, wherein the alteration software provides alteration that is permanent.

26. The medical device of claim 17, wherein the alteration mechanism provides alteration software, the alteration software supporting interface functionality.

27. The medical device of claim 26, wherein the alteration software includes a communication stack so as to support networking.

28. A medical imaging system, including a scope and one or more medical devices, the system comprising:
operational electronics associated with at least one medical device;
embedded software associated with operational electronics so as to determine function, in whole or in part, of a medical device; and,
an alteration mechanism associated with the operational electronics, the alteration mechanism providing alteration of at least one of operational electronics and embedded software.

29. The medical imaging system of claim 28, wherein the operational electronics supports a configurable component and the alteration mechanism provides alteration of the configurable component.

30. The medical imaging system of claim 29, wherein the operational electronics supports a programmable component and the alteration mechanism provides alteration of the programmable component.

31. The medical imaging system of claim 30, wherein the embedded software supports configuring software associated with the configurable component, and the alteration mechanism provides configuring alteration software that alters the configuring software.

32. The medical imaging system of claim 31, wherein the embedded software supports programming software associated with the programmable component, and the alteration mechanism provides programming alteration software that alters the programming software.

33. The medical imaging system of claim 29, wherein the embedded software supports configuring software associated with the configurable component, and the alteration mechanism provides configuring alteration software that alters the configuring software.

34. An imaging system, including a scope and one or more working devices, the system comprising:
operational electronics associated with at least one working device;
embedded software associated with operational electronics so as to determine function, in whole or in part, of a working device; and,
an alteration mechanism associated with the operational electronics, the alteration mechanism providing alteration of at least one of operational electronics and embedded software.

35. The imaging system of claim 34, wherein the operational electronics supports a configurable component and the alteration mechanism provides alteration of the configurable component.

36. The imaging system of claim 35, wherein the operational electronics supports a programmable component and the alteration mechanism provides alteration of the programmable component.

37. The imaging system of claim 36, wherein the embedded software supports configuring software associated with the configurable component, and the alteration mechanism provides configuring alteration software that alters the configuring software.

38. The imaging system of claim 37, wherein the embedded software supports programming software associated with the programmable component, and the alteration mechanism provides programming alteration software that alters the programming software.

39. The imaging system of claim 35, wherein the embedded software supports configuring software associated with the configurable component, and the alteration mechanism provides configuring alteration software that alters the configuring software.

40. The system of claim 28, wherein the medical device is a camera.

41. The system of claim 34, wherein the working device is a camera.
